(19) United States
(12) Patent Application Publication
(10) Pub. No.: US 2005/0176759 A1
(43) Pub. Date: Aug. 11, 2005

(54) 7-sulfonyl-3-benzazepine derivatives as modulators of the dopamine receptor and their use for the treatment cns disorders

(76) Inventors: Mahmood Ahmed, Harlow (GB); Steven Mark Bromidge, Verona (IT); Ian Thomson Forbes, Harlow (GB); Andrew Derrick Gribble, Harlow (GB); Christopher Norbert Johnson, Harlow (GB); Francis David King, Harlow (GB); Andrew P Lightfoot, Harlow (GB); Gregor James Macdonald, Harlow (GB); Stephen Frederick Moss, Harlow (GB); Mervyn Thompson, Harlow (GB); David R Witty, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/499,776

(22) PCT Filed: Dec. 20, 2002
(86) PCT No.: PCT/EP02/14824

(30) Foreign Application Priority Data
Dec. 21, 2001 (GB) 0130702.4
May 29, 2002 (GB) 0212398.2

(51) Int. Cl. A61K 31/47; A61K 31/4035

(52) U.S. Cl. 514/310; 514/416; 546/146

(57) ABSTRACT

Invented are non-peptide TPO mimetics. Also invented is a method of treating thrombocytopenia, in a mammal, including a human, in need thereof which comprises administering to such mammal an effective amount of a selected hydroxy-1-azobenzene derivative.

This invention relates to novel sulfone compounds having pharmacological activity, processes for their preparation, to compositions containing them and to their use in the treatment of CNS and other disorders such as psychotic disorders.

WO 98/27081, WO 99/02502, WO 99/37623, WO 99/42465 and WO 01/32646 (Smitline Beecham plc) disclose a series of aryl sulphonamide and sulphoxide compounds that are said to be 5-HT₆ receptor antagonists and which are claimed to be useful in the treatment of various CNS disorders. Grunewald, G. et al., (1999) J. Med. Chem. 42(1), 118-134 and Grunewald et al., (1999) 9(3), 481486 describe a series of 7-substituted 1,2,3,4-tetrahydroisoquinoline compounds (in particular 7-(phenylsulfonyl)-1,2,3,4-tetrahydroisoquinoline hydrochloride) as potent inhibitors of phenylethanolamine N-methyltnansferase (PNMT).

A structurally novel class of compounds has now been found which possess affinity for the 5-HT₆ receptor. The present invention therefore provides, in a first aspect, a compound of formula (I) or a pharmaceutically acceptable salt thereof: 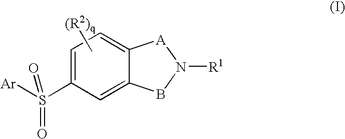
wherein:
R¹ represents hydrogen or C₁₋₆ alkyl;
A and B represent the groups —(CH₂)ₘ— and —(CH₂)ₙ—, respectively;
Ar represents a group —Ar¹ or a group —Ar²—Ar³;
each R² independently represents hydrogen, halogen, hydroxy, cyano, nitro, hydroxyC₁₋₆ alkyl, —CF₃, CF₃O—, C₁₋₆ alkyl, C₁₋₆ alkoxy, C₁₋₆alkanoyl, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S-C₁₋₆alkyl, —CO₂C₁₋₆alkyl, —CO₂NR⁵R⁶, —SO₂NR⁵R⁶, —(CH₂)ₚNR⁵R⁶, —(CH₂)NR⁵COR⁶, optionally substituted aryl ring, optionally substituted heteroaryl ring or optionally substituted heterocyclyl ring;
R⁵ and R⁶ each independently represent hydrogen, C₁₋₆alkyl or, together with the nitrogen or other atoms to which they are attached, form an azacycloalkyl ring or an oxo-substituted azacycloalkyl ring;
p and q independently represent an integer from 0 to 3;
m and n independently represent an integer of 1 or 2;
Ar¹ represents a naphthyl or bicyclic heteroaryl group each of which may be optionally substituted, wherein Ar¹ is attached to the sulphonyl moiety via a carbon atom;
Ar² represents an aryl or heteroaryl group each of which may be optionally substituted, wherein
Ar² is attached to the sulphonyl moiety via a carbon atom;
Ar³ represents an aryl or heteroaryl group, each of which may be optionally substituted;
Ar¹, Ar² and Ar³ may be optionally substituted by one or more substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, —CH₂)ₚC₃₋₆cycloalkyl, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆-alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group CONR^{3a}R^{3b} or SO₂NR^{3a}R^{3b}, wherein R^{3a} and R^{3b} independently represent hydrogen or C₁₋₆ alkyl or together may be fused to form a heterocyclyl or monocyclic heteroaryl group;
   or solvates thereof.

It is to be understood that the present invention covers all combinations of particular and preferred groups described herein above.

Alkyl groups, whether alone or as part of another group, may be straight chain or branched and the groups alkoxy and alkanoyl shall be interpreted similarly. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl. Alkyl moieties are more preferably C₁₋₄ alkyl, eg. methyl or ethyl. The term ‘halogen’ is used herein to describe, unless otherwise stated, a group selected from fluorine, chlorine, bromine or iodine. Preferred halogens are fluorine, chlorine and bromine.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy or hexyloxy.

As used herein, the term “cycloalkyl” refers to a non-aromatic hydrocarbon ring containing the specified number of carbon atoms. For example, C₃₋₇cycloalkyl means a non-aromatic ring containing at least three, and at most seven, ring carbon atoms. Examples of “cycloalkyl” as used herein include, but are not limited to, cyclopropyl, cyclobutyl, cyclopentyl, cyclohexyl and cycloheptyl. A C₆₋₇cycloalkyl group is preferred.

The term “aryl” includes phenyl and naphthyl.

The term “heteroaryl” is intended to mean a 5 or 6 membered monocyclic aromatic or a fused 8-10 membered bicyclic aromatic ring containing 1 to 3 heteroatoms selected from oxygen, nitrogen and sulphur.

The term “monocyclic heteroaryl” is intended to mean a 5 or 6 membered monocyclic aromatic ring containing 1 to 3 heteroatoms selected from oxygen, nitrogen and sulphur.

The term “bicyclic heteroaryl” is intended to mean a fused 8-10 membered bicyclic aromatic ring containing 1 to 3 heteroatoms selected from oxygen, nitrogen and sulphur.

Suitable examples of such monocyclic heteroaryl groups include thienyl, furyl, pyrrolyl, triazolyl, triazinyl, imidazolyl, oxazolyl, thiazolyl, oxadiazolyl, isothiazolyl, isoxazolyl, thiadiazolyl, pyrazolyl, pyrimidinyl, pyridazinyl, pyrazinyl and pyridyl. Suitable examples of such bicyclic heteroaryl groups include benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzoxadiazolyl, benzothiadiazolyl and the like. Heteroaryl groups, as described above, may be linked to the remainder of the molecule via a carbon atom or, when present, a suitable nitrogen atom except where otherwise indicated above.

As used herein, the term “heterocyclyl” refers to a 3- to 7-membered monocyclic saturated ring containing at least one heteroatom independently selected from oxygen, nitrogen and sulfur. Examples of suitable heterocyclic rings include, but are not limited to, piperidine and morpholine.

As used herein, the term “azacycloalkyl ring” refers to a 4- to 7-membered monocyclic saturated ring containing one nitrogen atom. Examples of suitable azacycloalkyl rings are azetidine, pyrrolidine, piperidine and hexahydroazepine.

As used herein, the term “oxo-substituted azacycloalkyl ring” refers to an azacycloalkyl ring as defined above substituted by one oxo group. Examples of suitable oxo-substituted azacycloalkyl rings include, but are not limited to, azetidinone, pyrrolidinone, piperidinone and azepinone.

As used herein, the term “substituted” refers to substitution with the named substituent or substituents, multiple degrees of substitution being allowed unless otherwise stated.

The present invention therefore also provides, in one aspect a compound of formula (IA) or a pharmaceutically acceptable salt thereof: 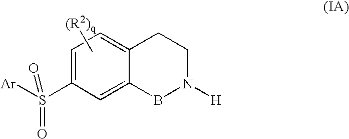
wherein:
Ar represents a group —Ar¹ or a group —Ar²—Ar³;
each R² independently represents hydrogen, halogen, cyano, —CF₃, CF₃O—, C₁₋₆ alkyl, C₁₋₆ alkoxy or C₁₋₆ alkanoyl;
q is as defined above;
B is as defined above;
Ar¹ and Ar² are as defined above;
Ar³ represents phenyl or a monocyclic heteroaryl group, each of which may be optionally substituted;
Ar¹, Ar² and Ar³ may be optionally substituted by one or more substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group CONR^{3c}R^{3d} or SO₂NR^{3c}R^{3d}, wherein R^{3c} and R^{3d} independently represent hydrogen or C₁₋₆ alkyl or together may be fused to form a 5- to 7-membered aromatic or non-aromatic heterocyclic ring optionally interrupted by an O or S atom;
   or solvates thereof

Preferably, A represents Ar¹.

Preferably, Ar¹, Ar² and Ar³ are substituted by 0 to 3 substituents, more preferably unsubstituted.

When Ar represents Ar¹, Ar¹ is preferably 2-naphthyl or 3-naphthyl or bicyclic heteroaryl (eg. quinolinyl or 1H-indolyl), most preferably 1H-indol-3-yl.

Preferably, B is (CH₂)₂.

Preferably, q is 0.

When Ar represents —Ar²Ar³, preferred embodiments where Ar² represents phenyl are described with reference to the compounds of formulae (IB) and (IC).

Thus, in a second embodiment of the invention, Ar represents —Ar²—Ar³ and Ar² represents phenyl i.e. a compound of formula (IB) 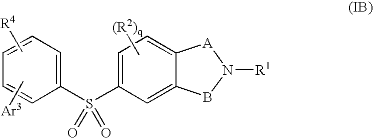
or a pharmaceutically acceptable salt or solvate thereof wherein groups A, B, R¹, R², q and Ar³ have any of the meanings as given hereinbefore and R⁴ represents hydrogen, hydroxy, C₁₋₆alkyl, C₁₋₆alkoxy, trifluoromethyl, trifluoromethoxy, halogen, —OSO₂CF₃, —CH₂)ₚC₃₋₆cycloalkyl, —C₁₋₆alkoxyC₁₋₆alkyl or —(CH₂)ₚOC₃₋₆cycloalkyl.

The R² groups may be located on any position on the phenyl ring.

Preferably, R¹ represents hydrogen or C₁₋₄alkyl. More preferably, R¹ represents hydrogen, methyl, ethyl, n-propyl or isopropyl. Even more preferably, R¹ represents hydrogen, methyl, ethyl or isopropyl.

Preferably, q represents 0 or 1.

When present, R² preferably represents hydrogen, halogen, C₁₋₆alkyl or C₁₋₆alkoxy. More preferably, R² represents hydrogen, halogen, C₁₋₄alkyl or C₁₋₄alkoxy. Even more preferably, R² represents hydrogen, methoxy or bromo.

When Ar represents —Ar²Ar³, Ar² preferably represents phenyl optionally substituted by chloro, fluoro, methoxy or cyano.

When Ar represents —Ar²Ar³, Ar³ preferably represents phenyl optionally substituted by hydrogen, C₁₋₄alkyl or C₁₋₄alkoxy.

Preferably, m and n both represent 2.

In compounds of formula (IB) in a first embodiment, when q represents 1, the R² group is located at the para-position relative to the group B i.e. a compound of formula (IB)^{a} 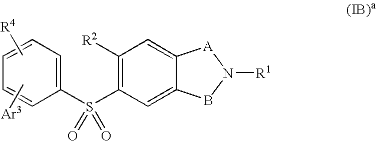
or a pharmaceutically acceptable salt or solvate thereof wherein groups A, B, R¹, R², Ar³ and R⁴ have any of the meanings as given hereinbefore.

When R² is located in the para-position i.e. compounds of formula (IB)^{a}, R² is preferably hydrogen or methoxy.

In a second embodiment of the embodiment of (IB), the Ar³ group is located at the meta-position relative to the sulfone group i.e. a compound of formula (IB)^{b} 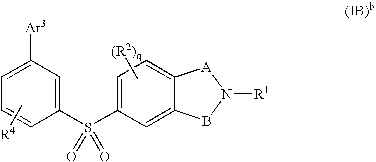
or a pharmaceutically acceptable salt or solvate thereof wherein groups A, B, R¹, R², q, Ar³ and R⁴ have any of the meanings as given hereinbefore.

When Ar³ is located in the meta-position i.e. compounds of formula (IB)^{b}, Ar³ is preferably phenyl. When Ar³ located in the meta-position is phenyl, the optional substituents on the phenyl ring are preferably chloro, fluoro, methoxy and cyano.

In a further embodiment of the embodiment of (IB), the Ar³ group is located at the para-position relative to the sulfone group i.e. a compound of formula (IB)^{c} 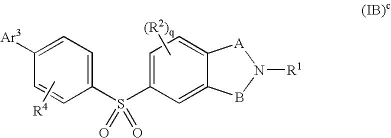
or a pharmaceutically acceptable salt or solvate thereof wherein groups A, B, R¹, R², q, Ar³ and R⁴ have any of the meanings as given hereinbefore.

When Ar³ is located in the para-position i.e. compounds of formula (IB)^{c}, Ar³ is preferably phenyl. When Ar³ located in the para-position is phenyl, the optional substituents on the phenyl ring are preferably chloro, fluoro, methoxy and cyano.

In a further embodiment of the embodiment of (IB), the R⁴ group is located at the para-position relative to the sulfone group i.e. a compound of formula (IB)^{d} 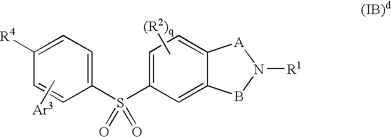
or a pharmaceutically acceptable salt or solvate thereof wherein groups A, B, R¹, R², q, Ar³ and R⁴ have any of the meanings as given hereinbefore.

When R⁴ is located in the para-position i.e. compounds of formula (IB)^{d}, R⁴ is preferably hydrogen or methyl.

In a further embodiment of the embodiment of (IB), the R⁴ group is located at the ortho-position relative to the sulfone group i.e. a compound of formula (IB)^{e} 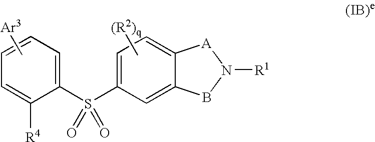
or a pharmaceutically acceptable salt or solvate thereof wherein groups A, B, R¹, R², q, Ar³ and R⁴ have any of the meanings as given hereinbefore.

When R⁴ is located in the ortho-position i.e. compounds of formula (IB)^{e}, R⁴ is preferably hydrogen or methoxy.

In a further embodiment of the embodiment of (IB), the Ar³ group is located at the meta-position relative to the sulfone group and the R⁴ group is located at the para-position relative to the sulfone group i.e. a compound of formula (IB)^{f} 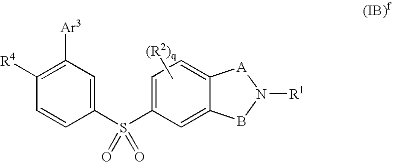
or a pharmaceutically acceptable salt or solvate thereof wherein groups A, B, R¹, R², q, Ar³ and R⁴ have any of the meanings as given hereinbefore.

In a further embodiment of the embodiment of (IB), the Ar³ group is located at the para-position relative to the sulfone group and the R⁴ group is located at the ortho-position relative to the sulfone group i.e. a compound of formula (IB)^{g} 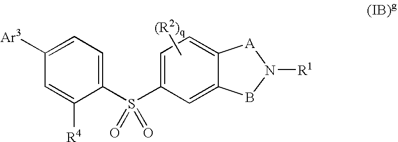
or a pharmaceutically acceptable salt or solvate thereof wherein groups A, B, R¹, R², q, Ar³ and R⁴ have any of the meanings as given hereinbefore.

In another embodiment of the embodiment of (IB), m is 2, n is 2 and q is 1, the R² group is located at the para-position relative to the group B, the R³ group is located at the meta-position relative to the sulfone group, the R⁴ group is located at the para-position relative to the sulfone group and the invention is a compound of formula (IB)^{h}: 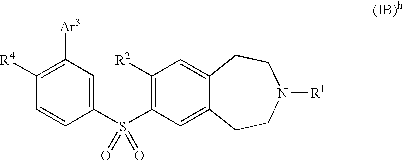
or a pharmaceutically acceptable salt or solvate thereof wherein groups R¹, R², Ar³ and R⁴ have any of the meanings as given hereinbefore.

In another embodiment of the embodiment of (IB), m is 2, n is 2 and q is 1, the R² group is located at the para-position relative to the group B, the Ar³ group is located at the meta-position relative to the sulfone group, the R⁴ group is located at the ortho-position relative to the sulfone group and the invention is a compound of formula (IB)ⁱ: 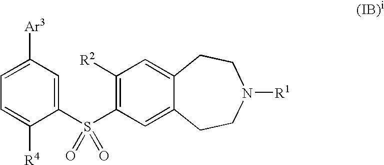
or a pharmaceutically acceptable salt or solvate thereof wherein groups R¹, R², Ar³ and R⁴ have any of the meanings as given hereinbefore.

In a further embodiment of the invention, the Ar³ group is located at the ortho-position relative to the sulfone group i.e. a compound of formula (IC) 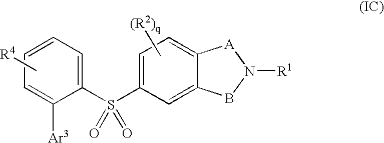
or a pharmaceutically acceptable salt or solvate thereof wherein groups A, B, R¹, R², q, Ar³ and R⁴ have any of the meanings as given hereinbefore.

For compounds of the formulae (I), (IA), (IB) and (IC), preferably, R⁵ and R⁶ independently represent hydrogen or C₁₋₄alkyl. More preferably, R⁵ and R⁶ independently represent hydrogen or methyl.

For compounds of the formula (I), (IA), (IB) and (IC) preferably, p represents 0.

For compounds of the formulae (I), (IA), (IB), or (IC), preferably, when R² represents an optionally substituted aryl, an optionally substituted heteroaryl, or an optionally substituted heterocyclyl, the optional substituents are independently selected from chloro, fluoro, bromo, methyl, ethyl, t-butyl, methoxy, trifluoromethyl, trifluoromethoxy, cyano and —S-methyl.

For compounds of the formulae (I), (IA), (IB), or (IC), preferably, Ar³ represents phenyl.

For compounds of the formulae (I), (IA), (IB), or (IC), preferably, when Ar³ represents an optionally substituted aryl or an optionally substituted heteroaryl, the optional substituents are independently selected from chloro, fluoro, bromo, methyl, ethyl, t-butyl, methoxy, trifluoromethyl, trifluoromethoxy, cyano and —S-methyl.

For compounds of the formulae (I), (IA), (IB), or (IC), preferably, when Ar³ represents phenyl, the optional substituents are independently selected from chloro, fluoro, bromo, methoxy, trifluoromethyl, trifluoromethoxy and cyano.

For compounds of the formulae (I), (IA), (IB) or (IC), preferably, R⁴ represents hydrogen, C₁₋₄alkyl or C₁₋₄alkoxy. More preferably, R⁴ represents hydrogen, methyl or methoxy.

In a further embodiment of the invention, m is 1 and n is 1 and the invention is a compound of formula (ID): 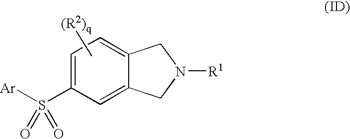
or a pharmaceutically acceptable salt or solvate thereof wherein groups R¹, R², q and Ar have any of the meanings as given hereinbefore.

In a further embodiment of the invention, m is 2 and n is 1 and the invention is a compound of formula (E): 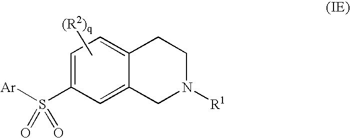
or a pharmaceutically acceptable salt or solvate thereof wherein groups R¹, R², q and Ar have any of the meanings as given hereinbefore.

In a further embodiment of the invention, m is 1 and n is 2 and the invention is a compound of formula (IF): 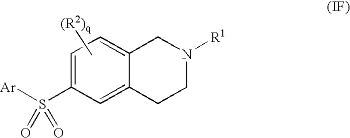
or a pharmaceutically acceptable salt or solvate thereof wherein groups R¹, R², q and Ar have any of the meanings as given hereinbefore.

In another embodiment of the invention, m is 2 and n is 2 and the invention is a compound of formula (IG): 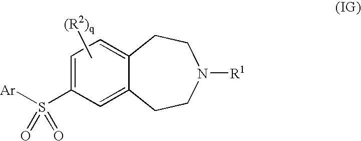
or a pharmaceutically acceptable salt or solvate thereof wherein groups R¹, R², q and Ar have any of the meanings as given hereinbefore.

Particular compounds according to the invention include those incorporated in Tables 1 and 2 and those specifically exemplified and named hereinafter including, without limitation:—
7-(6-Methyl-3-biphenylsulfonyl)-1,2,4,5-tetrahydro-3-benzazepine,
7-4′Cyano-3-biphenylsulfonyl)-1,2,4,5-tetrahydro-3-benzazepine;
7-(6-Methyl-3-biphenylsulfonyl)-3-methyl-1,2,4,5-tetrahydro-3-benzazepine;
7-3-(1H-indolyl)sulfonyl)-2,3,4,5-tetrahydro-1H-3-benzazepine;
7-(2-Phenyl)phenylsulfonyl-2,3,4,5-tetrahydro-1H-3-benzazepine;
   or a pharmaceutically acceptable salt thereof.

The compounds of the present invention may be in the form of their free base or physiologically acceptable salts thereof, particularly the monohydrochloride or monomesylate salts or pharmaceutically acceptable derivatives thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms.

The compounds of formula (I) may be prepared in crystalline or non-crystalline form, and, if crystalline, may optionally be solvated, eg. as the hydrate. This invention includes within its scope stoichiometric solvates (eg. hydrates) as well as compounds containing variable amounts of solvent (eg. water).

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (I) or a pharmaceutically acceptable salt thereof, which process comprises:
(a) reacting a compound of formula (II) 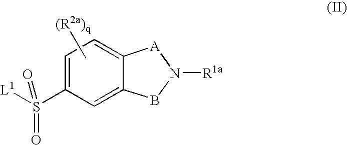
wherein R^{1a} and R^{2a} represent R¹ and R² as hereinbefore defined or are groups that may be readily convertible to R¹ and R², q is as defined above and L¹ represents a suitable leaving group (eg. a halogen atom such as chorine or fluorine); with a compound of formula Ar^{a}-M, wherein Ar^{a} is Ar as defined above or is optionally protected, and M represents a metal residue (eg. lithium or magnesium bromide) and thereafter, as necessary, deprotecting a protected derivative of a compound of formula (I); or
(b) reacting a compound of formula (II) as defined above with a compound of formula Ar^{a}—H, wherein Ar^{a} is as defined above; or
(c) reacting a compound of formula (III) 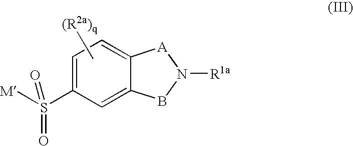
wherein R^{1a}, q and R^{2a} are as defined above and M′ represents a metal residue (eg. sodium); with a compound of formula Ar^{a}-L², wherein Ar^{a} is as defined above and L² represents a suitable leaving group (eg. a halogen atom such as chlorine or bromine) and thereafter, as necessary, deprotecting a protected derivative of a compound of formula (I); or
(d) deprotecting a compound of formula (I) which is protected; or
(e) interconversion to other compounds of formula (I).

Process (a) can be conveniently performed by mixing the two components at preferably −70° C. to room temperature in a suitable solvent such as tetrahydrofuran or ether for 10 minutes to 18 hours. Removal of certain R^{1a} protecting groups e.g. trifluoroacetyl, can also take place simultaneously during this process.

Process (b) typically comprises the use of a Lewis acid (eg. AlCl₃) and a suitable solvent such as chlorobenzene.

Process (c) typically comprises the use of a suitable solvent such as N,N-dimethylformamide and may optionally be performed in the presence of a copper salt such as copper (I) iodide at an elevated temperature, eg. 120° C.

In process (d), examples of protecting groups and the means for their removal can be found in T. W. Greene Protective Groups in Organic Synthesis' (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Process (e) may be performed using conventional interconversion procedures such as epimerisation, oxidation, reduction, alkylation, nucleophilic or electrophilic aromatic substitution, ester hydrolysis or amide bond formation. For example, indole N-methylation of a compound of Formula (I) where R¹ represents indolyl. Interconversion of one of the R^{1a}, R^{2a} or Ar^{a} groups to the corresponding R¹, R² or Ar groups typically arises when one compound of formula (I) is used as the immediate precursor of another compound of formula (I), or when it is easier to introduce a more complex or reactive substituent at the end of a synthetic sequence. For example, conversion of R^{1a} from a t-butoxycarbonyl (BOC) group to hydrogen is conducted by the treatment of the N-BOC protected compound with hydrogen chloride in ethanol or dioxan at room temperature.

Conversion of R^{1a} from hydrogen to an alkyl group is conducted by the treatment of the NH compound with the appropriate aldehyde in dichloroethane in the presence of a reducing agent, such as sodium triacetoxyborohydride, or by the treatment of the NH compound with the appropriate alkyl halide, such as iodomethane, under standard alkylation conditions (potassium carbonate in DMF at 60° C.).

The present invention also provides a general process (A) for preparing compounds of formula (I) wherein Ar represents —Ar²Ar³ and Ar² represents phenyl, which process comprises:
reacting a compound of formula (IV) 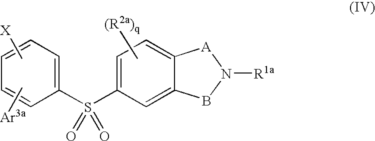
   with an aryl boronic acid of formula (V) 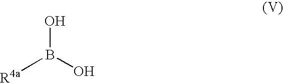
   wherein X is a leaving group, such as bromo, iodo, chloro, triflate or N₂⁺, A, B and q are as hereinbefore defined and R^{1a}, R^{2a}, Ar^{3a} and R^{4a} represent R¹, R², Ar³ and R⁴ as hereinbefore defined or are groups that may be readily convertible to R¹, R², Ar³ and R⁴. This general method (A) can be conveniently performed by mixing the two components in a suitable solvent such as toluene or ethanol containing aqueous sodium carbonate and a catalytic amount of Pd(PPh₃)₄ at room temperature or reflux under argon.

The present invention also provides a general process (B) for preparing compounds of formula (I) which process comprises:
reacting a compound of formula (VI) 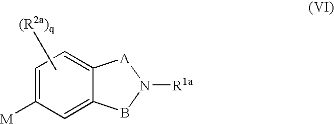
   with a compound of formula (VII) 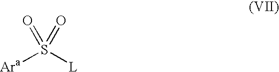
   wherein L is a leaving group, such as fluoro, chloro, alkoxy or aryloxy, M is a metal, such as lithium or magnesium, A, B and q are as hereinbefore defined and R^{1a}, R^{2a} and Ar^{a} represent R¹, R² and Ar as hereinbefore defined or are groups that may be readily convertible to R¹, R² and Ar. This general method (B) can be conveniently performed by mixing the two components at preferably −70° C. to room temperature in a suitable solvent such as tetrahydrofuran or ether for 10 minutes to 18 hours.

The present invention also provides a general process (C) for preparing compounds of formula (I) which process comprises:
reacting a reagent of formula (VIII) 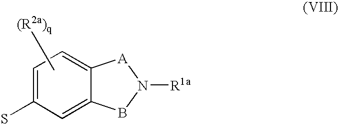
   with a compound of formula (IX) 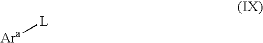
   followed by the oxidation of the resultant sulfide, by for example, meta-chloroperbenzoic acid, wherein L is a leaving group, such as fluoro, chloro, triflate or N₂⁺, A, B and q are as hereinbefore defined and R^{1a}, R^{2a} and Ar^{a} represent R¹, R² and Ar as hereinbefore defined or are groups that may be readily convertible to R¹, R² and Ar. This general method (C) can be conveniently performed by mixing the two components in a suitable solvent such as dimethylformamide, optionally at elevated temperature e.g. 120° C.

Compounds of formula (II) are known in the literature or may be prepared by known processes, for example, chlorosulfonation of the aromatic ring using chlorosulfonic acid. Conversion to the sulfonyl fluoride can be achieved, if required, by reaction with potassium fluoride in acetonitrile at room temperature. Suitable examples of an R^{1a} protecting group are trifluoroacetyl or the t-butoxycarbonyl (BOC) group.

Compounds of formula (III) may be prepared by reduction of a compound of formula (II) using a suitable reducing agent such as sodium sulfite in the presence of a base such as sodium carbonate or sodium bicarbonate in a suitable solvent system such as aqueous tetrahydrofuran. Where the compound of formula (III) is isolated as a free acid, deprotonation can be achieved by treatment with a base, eg. sodium hydride.

Compounds of formula (IV) may be prepared using a similar process to the process described in process (a) above.

Compounds of formula (V) are commercially available, or may be prepared by lithiation of the corresponding bromo aromatic compound, followed by quenching with tri-isopropyl borate then hydrolysis.

Compounds of formula (VI) may be prepared by metal halogen exchange using the corresponding bromo analogue as starting material and t-butyl lithium at low temperature.

Compounds of formula (VII) are commercially available or may be prepared by chlorosulfonylation of the aromatic ring. Conversion to the sulfonyl fluoride can be achieved, if required, by reaction with potassium fluoride in acetonitrile at room temperature.

Compounds of formula (VIII) may be prepared by reduction of compounds of formula (II) using for example lithium aluminium hydride in tetrahydrofuran. Deprotonation of the thiol can be achieved by treatment with base, e.g. sodium hydride.

Compounds of formula (IX) are commercially available or may be prepared using standard literature methodology.

Pharmaceutically acceptable salts may be prepared conventionally by reaction with the appropriate acid or acid derivative.

Compounds of formula (I), in particular compounds of formula (IA) and (IC) and their pharmaceutically acceptable salts have affinity for the 5-HT₆ receptor and are believed to be of potential use in the treatment of certain CNS disorders such as anxiety, depression, epilepsy, obsessive compulsive disorders, migraine, cognitive memory disorders (e.g. Alzheimers disease, age related cognitive decline and mild cognitive impairment), Parkinsons Disease, ADHD (Attention Deficit Disorder/Hyperactivity Syndrome), sleep disorders (including disturbances of Circadian rhythm), feeding disorders such as anorexia and bulimia, panic attacks, withdrawal from drug abuse such as cocaine, ethanol, nicotine and benzodiazepines, schizophrenia, and also disorders associated with spinal trauma and/or head injury such as hydrocephalus. Compounds of the invention are also expected to be of use in the treatment of certain GI (gastrointestinal) disorders such as IBS (Irritable Bowel Syndrome).

Thus the invention also provides a compound of formula (I) or a pharmaceutically acceptable salt thereof, for use as a therapeutic substance, in particular in the treatment or prophylaxis of the above disorders. In particular the invention provides for a compound of formula (I) or a pharmaceutically acceptable salt thereof, for use in the treatment of depression, anxiety, Alzheimers disease, age related cognitive decline, ADHD, obesity, mild cognitive impairment and schizophrenia.

Certain compounds of formula (I), in particular, compounds of formula (IB) and their pharmaceutically acceptable salts may also have affinity for the 5-HT_{2C} and 5-HT_{2A} receptors. These properties may give rise to anti-psychotic activity (e.g. improved effects on cognitive dysfunction) activity with reduced extrapyramidal side effects (eps), and/or anxiolytic/antidepressant activity. These could include, but are not limited to, attenuation of cognitive symptoms via 5-HT₆ receptor blockade (see Reavill, C. and Rogers, D. C., 2001, Investigational Drugs 2, 104-109), and reduced anxiety (see for example Kennett et al., Neuropharmacology 1997 April-May; 36 (4-5): 609-20), protection against EPS (Reavill et al., Brit. J. Pharmacol., 1999; 126:572-574) and antidepressant activity (Bristow et al., Neuropharmacology 39:2000; 1222-1236) via 5-HT_{2C} receptor blockade.

Certain compounds of formula (I), in particular, compounds of formula (IB) and their pharmaceutically acceptable salts have also been found to exhibit affinity for dopamine receptors, in particular the D₃ and D₂ receptors, and are useful in the treatment of disease states which require modulation of such receptors, such as psychotic conditions. Many of the compounds of formula (IB) have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable eps associated with many neuroleptic agents. Without wishing to be bound by theory, it has been suggested that blockade of the dopamine D₃ receptor may give rise to beneficial antipsychotic activity without significant eps (see for example Sokoloff et al, Nature, 1990; 347:146-151; and Schwartz et al, Clinical Neuropharmacology, Vol 16, No. 4, 295-314, 1993). Preferred compounds of the present invention are therefore those which have higher (e.g. ≧10×) affinity for dopamine D₃ than dopamine D₂ receptors (such affinity can be measured using standard methodology for example using cloned dopamine receptors—see herein).

Certain compounds of formula (I) may also exhibit affinity for other receptors not mentioned above, resulting in beneficial antipyschotic activity.

Certain compounds of formula (I), in particular, compounds of formula (IB) and their pharmaceutically acceptable salts are of use as antipsychotic agents for example in the treatment of schizophrenia, schizo-affective disorders, schizophreniform diseases, psychotic depression, mania, acute mania, paranoid and delusional disorders. Furthermore, they may have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D₃ receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyskinesias; depression; anxiety; agitation; tension; social or emotional withdrawal in psychotic patients; cognitive impairment including memory disorders such as Alzheirner's disease; psychotic states associated with neurodegenerative disorders, e.g. Alzheimer's disease; eating disorders; obesity; sexual dysfunction; sleep disorders; emesis; movement disorders; obsessive-compulsive disorders; amnesia; aggression; autism; vertigo; dementia; circadian rhythm disorders; and gastric motility disorders e.g. IBS.

Therefore, the invention provides a compound of formula (I) as hereinbefore described or a pharmaceutically acceptable salt or solvate thereof for use in therapy.

The invention also provides a compound of formula (I) and in particular a compound of formula (IB) or a pharmaceutically acceptable salt or solvate thereof for use in the treatment of a condition which requires modulation of a dopamine receptor.

The invention also provides a compound of formula (I) and in particular a compound of formula (IB) as hereinbefore described or a pharmaceutically acceptable salt or solvate thereof for use in the treatment of psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides the use of a compound of formula (I) and in particular a compound of formula (IB) as hereinbefore described or a pharmaceutically acceptable salt or solvate thereof in the manufacture of a medicament for the treatment of a condition which requires modulation of a dopamine receptor.

The invention also provides the use of a compound of formula (I) and in particular a compound of formula (IB) as hereinbefore described or a pharmaceutically acceptable salt or solvate thereof in the manufacture of a medicament for the treatment of psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides a method of treating a condition which requires modulation of a dopamine receptor, which comprises administering to a mammal in need thereof an effective amount of a compound of formula (I) and in particular a compound of formula (IB) as hereinbefore described or a pharmaceutically acceptable salt or solvate thereof.

A preferred use for dopamine antagonists according to the present invention is in the treatment of psychotic disorders, schizophrenia, Parkinsons disease, substance abuse, dyskinetic disorders, depression, bipolar disorder, anxiety and cognitive impairment.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment or prophylaxis of the above disorders.

In order to use the compounds of formula (I) in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or disable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 20.0 mg, for example 0.2 to 5 mg; and such unit doses may be administered more than once a day, for example two or three times a day, so that the total daily dosage is in the range of about 0.5 to 100 mg; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

Description 1

### 3-Trifluoroacetyl-1,2,4,5-tetrahydro-3-benzazepine-7-sulfonyl fluoride (D1)

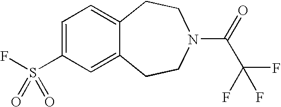

### a) 3-Trifluoroacetyl-1,2,4,5-tetrahydro-3-benzazepine-7-sulfonyl chloride

A solution of 3-trifluoroacetyl-1,2,4,5-tetrahydro-3-benzazepine (20 g, 80 mmol) in dichloromethane (50 ml) was added dropwise to a solution of chlorosulfonic acid (33 ml, 240 mmol) in more dichloromethane (200 ml) at 0° C. The resulting solution was stirred for 18 h without cooling then poured onto ice (250 g). The resulting organic layer was washed with brine (100 ml), dried (MgSO₄), and evaporated to give the subtitle compound as a white solid (23 g).

### b) 3-Trifluoroacetyl-1,2,4,5-tetrahydro-3-benzazepine-7-sulfonyl fluoride

A mixture of 3-trifluoroacetyl-1,2,4,5-tetrahydro-3-benzazepine-7-sulfonyl chloride (23 g, 67 mmol), potassium fluoride (12 g, 200 mmol), 18-crown-6 (0.1 g), and acetonitrile (100 ml) was stirred overnight. Water (200 ml) and ethyl acetate (200 ml) were added and the organic layer was washed with brine (100 ml), dried (MgSO₄), and evaporated to give the title compound as a white solid (21 g). ¹H NMR δ (d₆-DMSO) 3.2 (4H, m), 3.7 (4H, m), 7.6 (1H, m), and 8.0 (2H, m).

Description 2

### 3-Trifluoroacetyl-8-methoxy-1,2,4,5-tetrahydro-3-benzazepine-7-sulfonyl fluoride (D2)

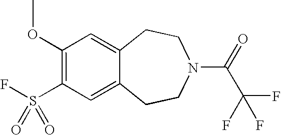

### a) 3-Trifluoroacetyl-8-methoxy-1,2,4,5-tetrahydro-3-benzazepine

To a mixture of 8-methoxy-1,2,4,5-tetrahydro-3-benzazepine hydrochloride (5.1 g, 25 mmol), triethylamine (8.4 ml, 60 mmol), and dichloromethane (100 ml) at 0° C., was added dropwise trifluoroacetic anhydride (3.5 ml, 26 mmol). The solution was stirred for 2 h without cooling then washed with saturated aqueous sodium hydrogen carbonate (100 ml), and water (100 ml), dried (MgSO₄), and evaporated to give the title compound as a white solid (5.5 g).

### b) 3-Trifluoroacetyl-methoxy-1,2,4,5-tetrahydro-3-benzazepine-7-sulfonyl chloride

Prepared from 3-triiluoroacetyl-8-methoxy-1,2,4,5-tetrahydro-3-benzazepine using the method of description 1(a), yield 85%.

### c) 3-Trifluoroacetyl-8-methoxy-1,2,4,5-tetrahydro-3-benzazepine-7-sulfonyl fluoride

Prepared from 3-trifluoroacetyl-8-methoxy-1,2,4,5-tetrahydro-3-benzazepine-7-sulfonyl chloride using the method of description 1(b), yield 80%.

¹H NMR δ (d₆-DMSO) 3.1 (4H, m), 3.7 (4H, m), 4.0 (3H, s), 7.3 (1H, 2s, rotamers), and 7.8 (1H, 2s, rotamers).

Description 3

### 7-(3-Trifluoromethysulfonyloxyphenylsulfonyl)-3-(t-butoxycarbony)-1,2,4,5-tetrahydro-3-benzazepine (D3)

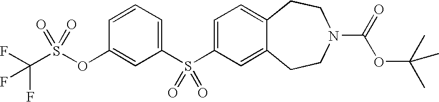

### a) 7-(3-t-Butyldimethysilyloxyphenylsulfonyl)-1,2,4,5-tetrahydro-3-benzazepine

Prepared from 3-trifluoroacetyl-1,2,4,5-tetrahydro-3-benzazepine-7-sulfonyl fluoride and 3-t-butyldimethylsilyloxybromobenzene using the method of example 1(b), yield 80%.

### b) 7-(3-t-Butyldimethysilyloxyphenylsulfonyl)-3-(t-butoxycarbonyl)-1,2,4,5-tetrahydro-3-benzazepine

A solution of 7-(3-t-butyldimethysilyloxyphenylsulfonyl)-1,2,4,5-tetrahydro-3-benzazepine (5.0 g, 12 mmol) in dichloromethane (100 ml) was treated with di-t-butyl dicarbonate (2.7 g, 12 mmol). After 30 min the solution was evaporated, and chromatography on silica, eluting with 10 to 50% ethyl acetate in hexane, gave the subtitle compound (5.4 g).

### c) 7-(3-Hydroxyphenylsulfonyl)-3-(t-butoxycarbonyl)-1,2,4,5-tetrahydro-3-benzazepine

7-(3-t-Butyldimethysilyloxyphenylsulfonyl)-3-(t-butoxycarbonyl)-1,2,4,5-tetrahydro-3-benzazepine (5.4 g, 10.5 mmol) was dissolved in a solution of tetra-n-butylammonium fluoride in THF (15 ml, 1M, 15 mmol). The solution was stirred for 1 h then diluted with ethyl acetate (100 ml) and washed with saturated aqueous sodium hydrogen carbonate (100 ml), and brine (100 ml), dried (MgSO₄), and evaporated. Chromatography on silica, eluting with 0 to 10% methanol in dichloromethane containing 0.1M ammonia, gave the subtitle compound (3.5 g).

### d) 7-(3-Trifuoromethysulfonyloxyphenylsulfonyl)-3-(t-butoxycarbonyl)-1,2,4,5-tetrahydro-3-benzazepine

A solution of 7-(3-hydroxyphenylsulfonyl)-3-(t-butoxycarbonyl)-1,2,4,5-tetrahydro-3-benzazepine (3.5 g) in dichloromethane 950 ml) at −20° C. was treated with triethylamine (1.4 ml, 10 mmol) and trifluoromethanesulfonic anhydride (1.5 ml, 9 mmol). The solution was stirred without cooling for 2 h then washed with saturated aqueous sodium hydrogen carbonate (50 ml), passed through a short silica plug, and evaporated. to give the title compound (4.3 g). ¹H NMR δ (d₆-DMSO) 1.3 (9H, s), 2.9 (4H, m), 3.4 (4H, m), 7.4 (1H, d, J=8 Hz), 7.8 (4H, m), and 8.1 (2H, m).

Description 4

### 3-(2′,2′,2′-Trichloroethyloxycarbonyl)-2,3,4,5-tetrahydro-1H-3-benzazepine (D4)

2,3,4,5-Tetrahydro-1-H-benzo[d]azepine [Deady, et al. J. Chem. Soc., Perkin Trans. 1 (1973), No. 8 782-3] (27 g, 0.184 mol) was dissolved in dichloromethane (400 ml) and treated with triethylamine (31 ml, 22.5 g, 0.22 mol) followed by the slow addition of 2,2,2-trichloroethoxychloroformate (28 ml, 43.1 g, 0.2 mol) maintaining the temperature around 25° C. with ice bath cooling. Once addition was complete the mixture was stirred for 1 h at RT then ice water (100 ml) added with stirring. The aqueous phase was separated and the organic phase washed with 5% aq hydrochloric acid (100 ml) and water (100 ml). The organic phase was dried with sodium sulphate, filtered and evaporated to give the title compound as a pink oil which crystallised slowly (57.6 g, 97%).

¹H NMR (CDCl₃) δ: 2.96 (4H, br d), 3.63-3.72 (4H, m), 4.81 (2H, s), 7.1-7.18 (4H, m).

Description 5

### 3-(2′,2′,2′-Trichloroethyloxycarbonyl)-2,3,4,5-tetrahydro-1H-3-benzazepine-7-sulfonyl chloride (D5)

Chlorosulphonic acid (75 ml) was cooled to 10° C. and treated with 3-(2′,2′,2′-trichloroethyloxycarbonyl)-2,3,4,5-tetrahydro-1H-3-benzazepine (D4) (57.5 g, 0.18 mol), added slowly over 30 minutes, with ice bath cooling to maintain the temperature below 20° C. The mixture was stirred for 16 hours then poured slowly onto a mixture of ice (400 g) and dichloromethane (150 ml) with vigorous stirring. The mixture was extracted into dichloromethane (2×100 ml), and the combined extracts washed with water (2×100 ml), filtered through celite and dried over sodium sulphate. The resulting solution was evaporated to give the title compound (D2) as an oil which rapidly crystallised (79.0 g, quantitative, with traces of solvents present).

¹H NMR (CDCl₃) δ: 3.10 (4H, br s), 3.71-3.76 (4H, m), 4.81 (2H, s), 7.38 (1H, t), 7.81-7.84 (2H, m).

Description 6

### 3-(2′,2′,2′-trichloroethyloxycarbonyl)-7-fluorosulfonyl-2,3,4,5-tetrahydro-1H-3-benzazepine (D6)

Potassium fluoride (1.8 g, 30.98 mmol) was added to a solution of crude 3-(2′,2′,2′-trichloroethyloxycarbonyl)-7-chlorosulfonyl-2,3,4,5-tetrahydro-1H-3-benzazepine (D5) (6.8 g, 16.78 mmol) in acetonitrile (30 ml). 18-crown-6 (0.068 g) was then added and the solution stirred for 18 h. The reaction mixture was partitioned between ethyl acetate (50 ml) and water (50 ml), and the aqueous layer then re-extracted with ethyl acetate (2×25 ml). The combined organic extracts were washed with brine (100 ml), dried (Na₂SO₄) and concentrated in vacuo to give an oil. Trituration with hexane gave the title compound (3.23 g) as an off-white solid.

¹H NMR (CDCl₃) δ: 3.09 (4H, br s), 3.69-3.76 (4H, m), 4.82 (2H, s), 7.38-7.42 (1H, t), 7.78-7.84 (2H, m)

Description 7

### 1-[7-(Biphenyl-2-sulfonyl)-1,2,4,5-tetrahydrobenzo[d]azepin-3-yl]-ethanone (D7)

To a suspension of 3-acetyl-2,3,4,5-tetydro-1H-benzo[d]azepine-7-sulfuric acid sodium salt (0.50 g, 1.82 mmol) in DMF (10 mL) was added 2-iodobiphenyl (0.25 g, 0.89 mmol) and the mixture heated to 120° C. for 20 minutes. After this period, copper iodide (0.35 g, 1.84 mmol) was added and the resulting brown mixture heated at 120° C. for 18 h. After allowing to cool to room temperature, saturated sodium hydrogencarbonate solution was added to provide a mixture with pH 9. This mixture was then extracted with dichlorornethane (3×50 mL), the organic phase washed with water (100 mL), dried (Na₂SO₄) and concentrated in vacuo. The crude material was purified by preparative HPLC to give the title compound (D7) as a colourless paste (20 mg).

¹H NMR (CDCl₃) δ: 2.18 (3H, s), 2.71 (2H, t), 2.68 (2H, t), 3.51 (2H, t), 3.64 (2H, t), 6.89 (1H, d), 6.96-7.01 (3H, m), 7.08 (1H, br s), 7.18-7.21 (3H, m), 7.29 (1H, d), 7.56-7.60 (2H, m), 8.42 (1H, d).

### EXAMPLE 1

### 7-(6-Methyl-3-biphenylsulfonyl)-1,2,4,5-tetrahydro-3-benzazepine (E1)

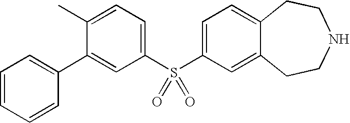

### a) 2-Methyl-5-bromobiphenyl

A mixture of 2-methyl-5-bromoaniline (1.8 g, 10 mmol) and hydrochloric acid (1 ml, 2M, 22 mmol) at 0° C. was diazotised with a solution of sodium nitrite (0.73 g, 10.5 mmol) in water (2 ml). When complete, aqueous tetrafluoroboric acid (2.0 ml, 48%, 11 mmol) was added and the resulting precipitate collected and washed with water, methanol, and ether to give the diazonium tetrafluoroborate (2.4 g).

This salt (2.0 g, 7 mmol) was then added in portions to a mixture of benzeneboronic acid (0.84 g, 7 mmol), palladium acetate (0.14 g, 0.7 mmol), and methanol (50 ml). When evolution of nitrogen had ceased, water (100 ml) and hexane (100 ml) were added. The organic layer was washed with brine (100 ml), dried (NgSO₄), and evaporated. Chromatography on silica, eluting with pentane, gave the subtitle compound (0.66 g).

### b) 7-(6-Methyl-3-biphenylsulfonyl)-1,2,4,5-tetrahydro-3-benzazepine

A solution of 2-methyl-5-bromobiphenyl (0.70 g, 2.8 mmol) in THF (10 ml) at −70° C. was treated with tert-butyllithium (3.2 ml, 1.7M in pentane, 5.5 mmol). After 20 min at −70° C., a solution of D1 (0.33 g, 11.0 mmol) in more THF (2 ml) was added, and after a further 30 min stirring without cooling, water (50 ml) and ethyl acetate (50 ml) were added. The organic layer was washed with brine (50 ml), dried (MgSO₄), and evaporated. Chromatography on silica, eluting with 0 to 15% methanol in dichloromethane containing 0.1M ammonia, gave the title compound isolated as the hydrochloride salt from ether (0.21 g). MH⁺ 378. ¹H NMR δ (d₆-DMSO) 2.3 (3H, s), 3.3 (8H, m), 7.4-7.9 (11H, m), and 9.2 (2H, bs).

### EXAMPLE 2

### 7-(4′Cyano-3-biphenylsulfonyl)-1,2,4,5-tetrahydro-3-benzazepine (E2)

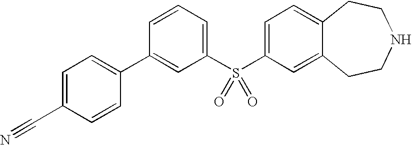

### a) 7-(4′Cyano-3-biphenylsulfonyl)-3-(t-butyloxycarbonyl)-1,2,4,5-tetrahydro-3-benzazepine

A mixture of 7-(3-trifluoromethylsulfonyloxyphenylsulfonyl)-3-t-butoxycarbonyl)-1,2,4,5-tetrahydro-3-benzazepine (0.43 g, 0.8 mmol), 4-cyanophenylboronic acid (0.18 g, 1.2 mmol), aqueous potassium carbonate (2.4 ml, 2M, 4.8 mmol), ethanol (2.5 ml), and toluene (10 ml) was degassed and then treated with tetrakis(triphenylphosphine)palladium (0) (50 mg). The solution was stirred for 3 h at 90° C. then cooled, diluted with ethyl acetate (10 ml) and washed with saturated aqueous sodium hydrogen carbonate (10 ml), and brine (10 ml), dried (MgSO₄), and evaporated. Chromatography on silica, eluting with 10 to 50% ethyl acetate in hexane, gave the subtitle compound (0.28 g).

### b) 7-(4′Cyano-3-biphenylsulfonyl)-1,2,4,5-tetrahydro-3-benzazepine

A solution of 7-(4′ cyano-3-biphenylsulfonyl)-3-(t-butyloxycarbonyl)-1,2,4,5-tetrahydro-3-benzazepine (0.28 g, 0.57 mmol) in ethanol (5 ml) was treated with hydrogen chloride in dioxan (5 ml, 4M, 20 mmol). After 4 h the solution was evaporated and the residue crystallised from ether to give the title compound as its hydrochloride salt (0.17 g). MH⁺ 389. ¹H NMR δ(d₆-DMSO) 3.2 (8H, m), 7.4 (1H, d, J=8 Hz), 7.8-8.2 (10H, m), and 9.2 (2H, bs).

### EXAMPLE 3

### 7-(6-Methyl-3-biphenylsulfonyl)-3-methyl-1,2,4,5-tetrahydro-3-benzazepine (E3)

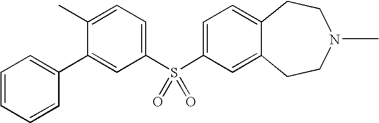

A mixture of E1 hydrochloride salt (0.14 g, 0.34 mmol), sodium triacetoxyborohydride (0.4 g), aqueous formaldehyde (0.4 ml, 37%), and 1,2-dichloroethane (10 ml) was stirred for 18 h then diluted with dichloromethane (50 ml) and washed with saturated aqueous sodium hydrogen carbonate (50 ml), dried (MgSO₄), and evaporated to give the title compound isolated as the hydrochloride salt from ether (0.11 g). MH⁺ 392. ¹H NMR δ(d₆-DMSO) 2.3 (3H, s), 2.8(3H, d J=5 Hz), 3.0-3.6(8H, m), 7.4-7.9 (11H, m), and 11.1 (1H, bs).

Examples 4-42 were prepared using analogous procedures to Examples 1, 2 and 3. Products were isolated as either the free bases or hydrochloride salts. All ¹H NMR are consistent with the structures shown.

TABLE 1

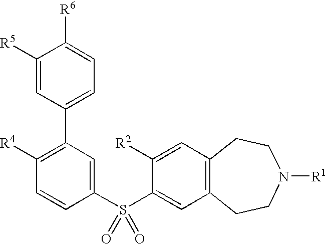

| Example | R¹ | R² | R⁴ | R⁵ | R ⁶ | MH⁺ |
|---|---|---|---|---|---|---|
| 4 | Me | H | H | H | H | 378 |
| 5 | H | OMe | H | H | H | 394 |
| 6 | H | H | H | H | Cl | 398 |
| 7 | Me | H | H | H | Cl | 412 |
| 8 | H | H | OMeH | H | 394 | |
| 9 | iPr | H | H | H | H | 406 |
| 10 | H | H | H | H | CN | 389 |
| 11 | H | H | H | CN | H | 389 |
| 12 | H | H | H | F | H | 383 |
| 13 | H | H | H | OMe | H | 394 |
| 14 | H | H | H | H | F | 382 |
| 15 | H | H | H | H | OMe | 394 |
| 16 | H | H | H | Cl | H | 398 |
| 17 | Me | H | H | H | F | 396 |
| 18 | Me | H | H | H | CN | 403 |
| 19 | Me | H | H | H | OMe | 408 |
| 20 | Me | H | H | F | H | 396 |
| 21 | Me | H | H | Cl | H | 412 |
| 22 | Me | H | H | CN | H | 403 |
| 23 | Me | H | H | OMe | H | 408 |
| 24 | Me | H | OMeH | H | 408 | |
| 25 | Me | H | Me | H | H | 392 |
| 26 | H | H | Me | H | H | 378 |
| 27 | Me | MeO | H | H | Cl | 442 |
| 28 | Me | MeO | H | H | F | 426 |

TABLE 2

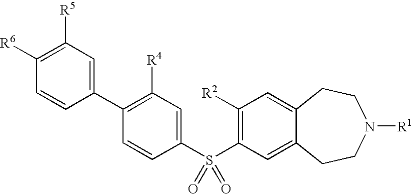

| Example | R¹ | R² | R⁴ | R⁵ | R⁶ | MH⁺ |
|---|---|---|---|---|---|---|
| 29 | H | H | H | H | Cl | 398 |
| 30 | Me | H | H | H | Cl | 412 |
| 31 | H | H | H | H | CN | 389 |
| 32 | H | H | H | H | OMe | 394 |
| 33 | H | H | H | F | H | 382 |
| 34 | H | H | H | Cl | H | 398 |
| 35 | H | H | H | CN | H | 389 |
| 36 | H | H | H | OMe | H | 394 |
| 37 | Me | H | H | H | OMe | 408 |
| 38 | Me | H | H | F | H | 396 |
| 39 | Me | H | H | Cl | H | 412 |
| 40 | Me | H | H | CN | H | 403 |
| 41 | Me | H | H | OMe | H | 408 |
| 42 | Me | MeO | H | H | Cl | 442 |

### EXAMPLE 43

### 7-(3-(1H-indolyl)sulfonyl)-2,3,4,5-tetrahydro-1H-3-benzazepine (E43)

^{t}BuLi (1.7 M in pentane, 5.7 mL) was added slowly to a solution of 3-bromo-1-(t-butyldimethylsilyl)indole (1.5 g, 4.83 mmol) at −78° C., under argon. After stirring for 5 min., 3(2′,2′,2′-trichloroethyloxycarbonyl)-7-fluorosulfonyl-2,3,4,5-tetrahydro-1H-3-benzazepine (D6) (1.96 g, 5.04 mmol) was added as a solution in THF (15 mL). The solution was stirred at −78° C. for 30 min., then at room temperature for 90 min. The resulting mixture was then poured into a saturated solution of NH₄Cl (50 mL) and extracted with DCM (2×50 mL). The organic layer was washed with brine (100 mL), dried (Na₂SO₄) and concentrated in vacuo to give a brown oil. Purification by column chromatography (DCM/MeOH) gave the title compound as a brown solid.

¹H NMR (DMSO) δ:2.81 (4H, m), 2.88-2.92 (4H, m), 7.18-7.30 (3H, m), 7.48-7.50 (1H, d), 770-7.79 (3H, m), 8.14 (1H, m), 12.25 (1H, br s)

### EXAMPLE 44

### 7-(3-(1H-indolyl)sulfonyl)-2,3,4,5-tetrahydro-1H-3-benzazepine hydrochloride (E44)

7-(3-Sulfonyl-1H-indolyl)-2,3,4,5-tetrahydro-1H-3-benzazepine (E43) (59 mg, 0.18 mmol) was taken up in methanol (5 mL) and 1M ethereal HCl (0.19 mL, 0.19 mmol) added. Solution concentrated to yield the title compound as a pale brown solid (64 mg). Mass Spectrum (API⁺) 327 (MH⁺).

### EXAMPLE 45

### 7-(2-Phenyl)phenylsulfonyl-2,3,4,5-tetrahydro-1H-3-benzazepine hydrochloride (E45)

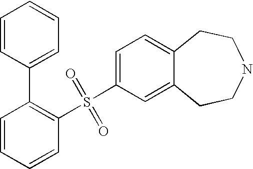

A solution of 1-[7-biphenyl-2-sulfonyl)-1,2,4,5-tetrahydrobenzo[d]azepin-3-yl]-ethanone (D7) (20 mg, 0.05 mmol) in n-butanol (1 mL) and 3M HCl (2 mL) was heated at reflux for 9 h. After this period, the mixture was allowed to cool to ambient temperature and concentrated in vacuo to afford the title compound (E45) (15 mg) as a pale yellow solid.

¹H NMR (CD₃OD) δ: 2.94-2.96 (2H, m), 3.11 (2H, br s), 3.18-3.25 (4H, m), 6.96-6.98 (3H, m), 7.14 (1H, d), 7.16 (1H, d), 7.21-7.26 (3H, m), 7.35 (1H, t), 7.65-7.72 (2H, m), 8.39 (1H, d).

Mass Spectrum (API⁺) 364 (MH⁺).

Examples 46-48 were prepared using analogous procedures to Examples 1, 2 and 3. Products were isolated as either the free bases or hydrochloride salts. All ¹H NMR are consistent with the structures shown.

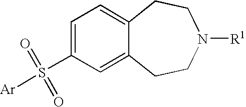

| Example | Ar | [MH] |
|---|---|---|
| 46 | 1-naphthyl | 338 |
| 47 | 2-naphthyl | 338 |
| 48 | 8-quinolinyl | 339 |

Pharmacological Data
Biological Test Methods
Binding Experiments on Cloned Dopamine (e.g. D2 and D3) Receptors

The ability of the compounds to bind selectively to human D2/D3 dopamine receptors can be demonstrated by measuring their binding to cloned receptors. The inhibition constants (Kᵢ) of test compounds for displacement of [¹²⁵I]-Iodosulpride binding to human D2/D3 receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centriflgation. Cell pellets were stored frozen at −80° C. Crude cell membranes were prepared by homogenisation followed by high-speed centrifugation, and characterisation of cloned receptors achieved by radioligand binding.

Preparation of CHO cell membranes: Cell pellets were gently thawed at room temperature, and resuspended in about 20 volumes of ice-cold Extraction buffer; 5 mM EDTA, 50 mM Trizina pre-set crystals (pH7.4@37° C.), 1 mM MgCl₂, 5 mM KCl and 120 mM NaCl. The suspension was homogenised using an Ultra-Turrax at full speed for 15 seconds. The homogenate was centrifliged at 18,000 r.p.m for 15 min at 4° C. in a Sorvall RC5C centrifuge. Supernatant was discarded, and homogenate re-suspended in extraction buffer then centrifugation was repeated. The final pellet was resuspended in 50 mM Trizma pre-set crystals (pH 7.4@37° C.) and stored in 1 ml aliquot tubes at −80° C. (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding experiments: Crude D2/D3 cell membranes were incubated with 0.03 nM [¹²⁵I]-Iodosulpride (˜2000 Ci/mmol; Amersham, U. K., and the test compound in a buffer containing 50 mM Trizma pre-set crystals (pH 7.4 @37° C.), 120 mM NaCl, 5 mM KCl, 2 mM CaCl₂, 1 mM MgCl₂, 0.3% (w/v) bovine serum albumin. The total volume is 0.2 ml and incubated in a water bath at 37° C. for 40 minutes. Following incubation, samples were filtered onto GF/B Unifilters using a Can berra Packard Filtermate, and washed four times with ice-cold 50 mM Trizma pre-set crystals (pH 7.4 @37° C.). The radioactivity on the filters was measured using a Can berra Packard Topcount Scintillation counter. Non-specific binding was defined with 10 μM SKF-102161 (YM-09151). For competition curves, 10 serial log concentrations of competing cold drug were used (Dilution range: 10 μM-10 pM). Competition curves were analysed using Inflexion, an iterative curve fitting programme in Excel. Results were expressed as pKᵢ values where
pKᵢ=−log10[Ki].

The exemplified compounds have pKᵢ values within the range of 5.8-8.0 at the dopamine D₃ receptor.

The exemplified compounds have pKᵢ values within the range of 5.3-4.6 at the dopamine D₂ receptor.

Binding Experiments on Cloned 5-HTA Receptors

Compounds can be tested following the procedures outlined in WO 98/27081.

The exemplified compounds have pKᵢ values within the range of 6.7-10.0 at the serotonin 5-HT₆ receptor. More particularly, the compounds of Examples 43 and 44 have pKᵢ values within the range of 9.0-10.0.

Binding Experiments on Cloned 5-HT_{2A} and 5-HT_{2C} receptors

Compounds can be tested following the procedures outlined in WO 94/04533.

The exemplified compounds have pKi values within the range of 6.4-9.0 at the serotonin 5-HT_{2C} receptor and 5.9-8.6 at the serotonin 5-HT_{2A} receptor.

1. A compound of formula (I) or a pharmaceutically acceptable salt thereof: 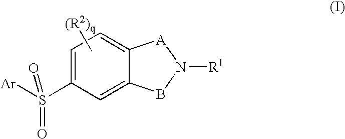
wherein:
R¹ represents hydrogen or C₁₋₆ alkyl;
A and B represent the groups —(CH₂)ₘ— and —(CH₂)ₙ—, respectively;
Ar represents a group —Ar¹ or a group —Ar²—Ar³;
each R² independently represents hydrogen, halogen, hydroxy, cyano, nitro, hydroxyC₁₋₆ alkyl, —CF₃, CF₃O—, C₁₋₆ alkyl, C₁₋₆ alkoxy, C₁₋₆ alkanoyl, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁵R⁶, —SO₂NR⁵R⁶, —(CH₂)ₚNR⁵R⁶, —(CH₂)ₚNR⁵COR⁶, optionally substituted aryl ring, optionally substituted heteroaryl ring or optionally substituted heterocyclyl ring;
R⁵ and R⁶ each independently represent hydrogen, C₁₋₆alkyl or, together with the nitrogen or other atoms to which they are attached, form an azacycloalkyl ring or an oxo-substituted azacycloalkyl ring;
p and q independently represent an integer from 0 to 3;
m and n independently represent an integer of 1 or 2;
Ar¹ represents a naphthyl or bicyclic heteroaryl group each of which may be optionally substituted, wherein Ar¹ is attached to the sulphonyl moiety via a carbon atom;
Ar² represents an aryl or heteroaryl group each of which may be optionally substituted, wherein Ar² is attached to the sulphonyl moiety via a carbon atom;
Ar³ represents an aryl or heteroaryl group, each of which may be optionally substituted;
Ar¹, Ar² and Ar³ may be optionally substituted by one or more substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, —(CH₂)ₚC₃₋₆cycloalkyl, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, arylsulfonyl, arylsulfonyloxy, arylsulfonylC₁₋₁₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group CONRR^{3a}R^{3b} or SO₂NR^{3a}R^{3b}, wherein R^{3a} and R^{3b} independently represent hydrogen or C₁₋₆ alkyl or together may be fused to form a heterocyclyl or monocyclic heteroaryl group;
or solvates thereof.

2. A compound of formula (IA) or a pharmaceutically acceptable salt thereof: 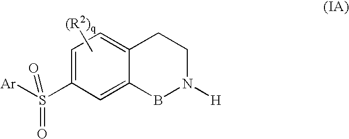
wherein:
Ar represents a group —Ar¹ or a group —Ar²—Ar³;
each R² independently represents hydrogen, halogen, cyano, —CF₃, CF₃O—, C₁₋₆ alkyl, C₁₋₆ alkoxy or C₁₋₆ alkanoyl;
q is as defined in claim 1;
B is as defined in claim 1;
Ar¹ and Ar² are as defined in claim 1;
Ar³ represents phenyl or a monocyclic heteroaryl group, each of which may be optionally substituted;
Ar¹, Ar² and Ar³ may be optionally substituted by one or more substituents which may be the same or different, and which are selected from the group consisting of halogen, hydroxy, cyano, nitro, trifluoromethyl, trifluoromethoxy, C₁₋₆ alkyl, trifluoromethanesulfonyloxy, pentafluoroethyl, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆alkylsulfonylC₁₋₆ alkyl, arylsulfonyl, arylsulfonyloxy, arylsulfonylC₁₋₆ alkyl, C₁₋₆ alkylsulfonamido, C₁₋₆ alkylamido, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group CONR^{3c}R^{3d} or SO₂NR^{3c}R^{3d}, wherein R^{3c} and R^{3d} independently represent hydrogen or C₁₋₆ alkyl or together may be fused to form a 5- to 7-membered aromatic or non-aromatic heterocyclic ring optionally interrupted by an O or S atom;
or solvates thereof.

3. A compound of formula (IB) or a pharmaceutically acceptable salt thereof: 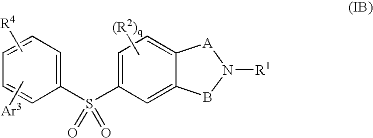
wherein groups A, B, R¹, R², q and Ar³ are as defined in claim 1 and R⁴ represents hydrogen, hydroxy, C₁₋₆alkyl, C₁₋₆alkoxy, trifluoromethyl, trifluoromethoxy, halogen, —OSO₂CF₃, —(CH₂)ₚC₃₋₆cycloalkyl, —C₁₋₆alkoxyC₁₋₆alkyl or —(CH₂)ₚOC₃₋₆cycloalkyl.

4. A compound as defined in claim 1 which is
7-(6-Methyl-3-biphenylsulfonyl)-1,2,4,5-tetrahydro-3-benzazepine,
7-(4′Cyano-3-biphenylsulfonyl)-1,2,4,5-tetrahydro-3-benzazepine;
7-(6-Methyl-3-biphenylsulfonyl)-3-methyl-1,2,4,5-tetrahydro-3-benzazepine;
7-(3-(1H-indolyl)sulfonyl)-2,3,4,5-tetrahydro-1H-3-benzazepine;
7-(2-Phenyl)phenylsulfonyl-2,3,4,5-tetrahydro-1H-3-benzazepine;
or a pharmaceutically acceptable salt thereof.

5. A pharmaceutical composition comprising a compound as defined in claim 1 or a pharmaceutically acceptable salt thereof and a pharmaceutically acceptable carrier therefor.

6. A method of treating depression, anxiety, Alzheimers disease, age related cognitive decline, ADHD, obesity, mild cognitive impairment and schizophrenia which comprises administering a safe and therapeutically effective amount to a patient in need thereof of a compound as defined in claim 1 or a pharmaceutically acceptable salt thereof.

7. A method of treating a condition which requires modulation of a dopamine receptor, which comprises administering to a mammal in need thereof an effective amount of a compound of formula (I) or a pharmaceutically acceptable salt or solvate thereof as defined in claim 1.

8. A method of treating psychotic disorders, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia and circadian rhythm disorders, which comprises administering to a mammal in need thereof an effective amount of a compound of formula (I) or a pharmaceutically acceptable salt or solvate thereof as defined in claim 1.
